(19) United States
(12) Patent Application Publication
(10) Pub. No.: US 2003/0093151 A1
(43) Pub. Date: May 15, 2003

(54) Implantable mammary prosthesis with flexible sheet

(75) Inventor: Jianjun Zhang, Guangzhou (CN)

Correspondence Address: CROWELL & MORING LLP, INTELLECTUAL PROPERTY GROUP, P.O. BOX 14300, WASHINGTON, DC, 20044-4300, US

(73) Assignee: Guangzhou Wanhe Material Co. Ltd.

(21) Appl. No.: 10/322,618

(22) Filed: Dec. 19, 2002

Related U.S. Application Data
(63) Continuation of application No. PCT/CN01/01638, filed on Dec. 21, 2001.

(30) Foreign Application Priority Data
Dec. 22, 2000 (CN) 00130876.9

(51) Int. Cl. A61F 2/12

(52) U.S. Cl. 623/8

(57) ABSTRACT

A surgical implantable mammary prosthesis adapted to resist capsular contracure comprises a flexible sac having a generally circular base and containing an appropriate gel or fluid, and a flexible sheet extending outwardly from said circular base and being peripherally integral with said circular base and forming a ring around said circular base. The unique design in this invention can effectively prevent the implanted mammary prosthesis from displacement, deformation and capsular contracture after breast augmentation. It can also be readily removed when desired by selecting suitable number of holes or of layers.

### CROSS REFERENCE TO RELATED APPLICATIONS

The present application is a continuation of International Patent Application No. PCT/CN01/01638, filed Dec. 21, 2001, designating the United States of America, the entire disclosure of which is incorporated herein by reference. Priority is claimed based on Chinese Patent Application No. 00130876.9, filed Dec. 22, 2000.

### BACKGROUND OF THE INVENTION

1. Field of the Invention

This invention relates to an implantable mammary prosthesis, and more particularly to an implantable mammary prosthesis which comprises a flexible sac and a flexible sheet.

2. Description of the Prior Art

Two categories of implantable mammary prostheses are widely used at present: the prostheses which contain a silicone gel and prostheses which contain physiological saline. Regardless which category of prostheses is implanted, implantation leads to a foreign body reaction in the organism and this reaction will lead to the formation of a living fibrous capsula which completely surrounds the implanted prosthesis. Therefore, there are some disadvantages related to the prior art. The prosthesis may shift or be displaced due to the gravity or outside forces, and the gel or liquid filled in it may move around inside it. This will cause the prosthesis deform temporarily. Furthermore, as a normal tissue reaction to the presence of a foreign body, the scar tissue around the prosthesis will undergo contraction during the healing process, which is also known as the spherical capsular contracture of the prosthesis. As the tissue around the prosthesis contracts the fixed volume of the silicone gel or the physiological saline within the prostheses is forced into a shape having the smallest possible surface area, i.e. a sphere surrounded essentially by scar tissue. At the same time, the connective tissue will grow within the space which has been released by the prosthesis and occupy it. As a result, the temporary deformation and shift of the prosthesis will gradually develop into a permanent deformation and shift. The inner surface area of the fibrous capsula will decrease substantially. Then the fibrous capsula produces a higher pressure on the prosthesis and the pressure within the prosthesis will increase greatly. Thus, the resulting hard spherical prosthesis results in an aesthetically undesirable breast.

U.S. Pat. No. 4,902,294 to Gosserez provides an implantable mammary prosthesis comprising a flexible pocket having a generally circular base and being dome shaped when filled with an appropriate fluid, and a substantially flat flexible sheet. The circular base and the flexible sheet may be made from the same surgical grade biocompatible film material. A flexible peripheral flange formed by the said flexible sheet extends outwardly from the circular base a distance equal to at least one fifth the radius of circular base. In U.S. Pat. No. 4,902,294, the flexible sheet is designed to avoid the formation of a spherical capsular contracture. But the disadvantage of such a design is that the said flexible sheet is liable to fold and can not fix the prosthesis in a satisfactory way. Once it folds or the prosthesis moves it will no longer be able to combat capsular contracture.

U.S. Pat. No. 4,963,150 to Brauman provides an implantable prosthetic device comprising a flexible container and a soft gel or liquid filling the flexible container. An outer plastic covering made of rough textured silicone is bonded to the flexible container. The outer plastic covering having a rough textured external surface provides a multiplicity of pores and/or interstices, and encases the flexible container in order to prevent the contraction of scar tissue around the prosthetic device from pulling the prosthetic device into a hard, spherical shape. But one disadvantage of such a prosthetic device is that it could not decrease substantially the capsular contracture in practical usage. Another disadvantage is that a multiplicity of pores and/or interstices on said covering may increase the operational difficulty when such a prosthetic device is removed from the body in some circumstances.

Thus, a satisfactory solution to combat the formation of a retractile fibrous capsula has not heretofore been found. And it is also very important that the prosthesis can be readily removed from the human body when an undesirable reaction to the prosthesis occurs or should total removal be desired for whatever reason.

### SUMMARY OF THE INVENTION

It is, therefore, the principal object of this invention to provide an implantable mammary prosthesis which resists the force of encapsulating tissue sufficiently to avoid formation of a spherical capsular contracture.

It is another object of this invention to provide an implantable mammary prosthesis which can readily be removed from the human body without encountering any untoward effects.

These and other objects of the present invention can be accomplished by an improved implantable mammary prosthesis adapted to resist capsular contracure, comprising a flexible sac having a generally circular base and being dome shaped when filled with an appropriate gel or fluid, and a flexible sheet extending outwardly from said circular base and being peripherally integral with said circular base and forming a ring around said circular base. The improvement of the invention is that a multiplicity of holes is provided on said flexible sheet.

The inventor has noticed that the newly formed tissue through the holes of the flexible sheet can prevent the prosthesis from shifting and the sheet from folding. Therefore, a better effect to combat the spherical contracture can be observed as compared with those prostheses which do not have a multiplicity of holes on said flexible sheet.

Other characteristics and the advantages of the invention will emerge from the following detailed description of this invention.

### BRIEF DESCRIPTION OF THE DRAWINGS

The invention is described below with reference to the drawing in which:

FIG. 1 shows an implantable mammary prosthesis having a flexible sac and a flexible sheet with a multiplicity of holes.

### DETAILED DESCRIPTION OF THE INVENTION

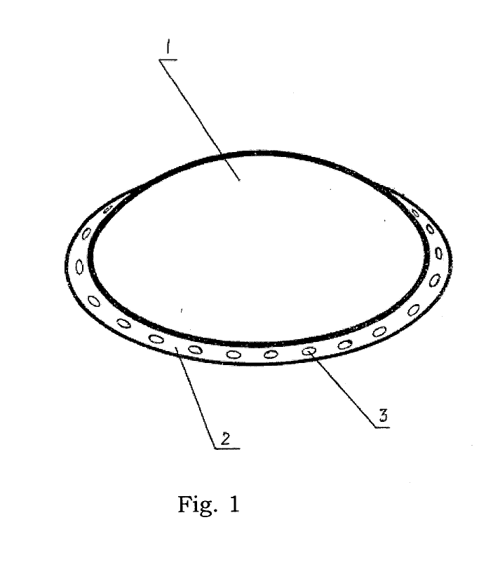

Referring now to FIG. 1 there is shown an implantable mammary prosthesis which includes a flexible sac 1 containing filler material. Sac 1 has a generally circular base, and a flexible sheet 2 extends outwardly from said circular base and is peripherally integral with said circular base and thus forms a ring around said circular base. A multiplicity of holes 3 are provided on said flexible sheet 2. When said prosthesis is implanted in human body the tissue can grow through the holes 3, which make said prosthesis fixed very well and thus prevent it from shifting.

The width of sheet 2, which is linked to said circular base of an implantable prosthesis, is preferably between about 2 and 25 mm, more preferably about 10 mm. The main function of sheet 2 is to prevent body tissue from growing towards sac 1 and thus to prevent the prosthesis from deforming due to the capsular contracture and pressure of body tissue.

When human body's position changes from lying state to standing state the implanted prosthesis may prolapse or shift. In order to prevent its displacement or overturn, a multiplicity of holes 3 are provided on sheet 2. The number of holes 3 can be determined according to specific cases. If the prosthesis will not be removed after being implanted the number can be increased to some extent; and vice versa, the number can be decreased for ease of removing the prosthesis. The gap or distance between two adjacent holes 3 is from about 5 mm to about 40 mm, more preferably from about 10 mm to about 15 mm. The sectional shape of the holes 3 can be a circle, a rectangle, an ellipse, or any other possible shapes. If the holes 3 are in the shape of a circle their radii are in the range of about 1 mm to about 10 mm, more preferably about 2 mm to 6 mm.

As for this invention, sheet 2 of said prosthesis can be made up of single layer or several layers. If sheet 2 is made up of several layers these layers can be connected with each other or separated from each other. A better fixation can be obtained for a prosthesis with a sheet made up of several layers even if there are not any holes on this sheet, because the body tissue can grow between these layers. The number of the layers is unlimited in principle. But the preferable number is between 2 and 10. If the prosthesis will not be removed after being implanted the number can be increased to some extent, for example more than 4; conversely, the number can be decreased for ease of removing the prosthesis, for example less than 3.

The filler material in sac 1 can be silicone gel, physiological saline or any other appropriate gel or liquid. The material of which sac 1 or sheet 2 is made can be silicone elastomer or any other surgical grade biocompatible material.

The unique design in this invention can effectively prevent the implanted mammary prosthesis from displacement, deformation and capsular contracture after breast augmentation. It can also be readily removed when desired by selecting suitable number of holes or of layers.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations falling within the scope of the appended claims and equivalents thereof.

What is claimed is:

1. A surgical implantable mammary prosthesis adapted to resist capsular contracture, comprising (1) a flexible sac having a generally circular base and containing an appropriate gel or fluid, and (2) a flexible sheet extending outwardly from said circular base and being peripherally integral with said circular base and forming a ring around said circular base, wherein the flexible sheet comprises a multiplicity of holes.

2. The mammary prosthesis of claim 1, wherein the width of said sheet is from about 2 mm to about 25 mm.

3. The mammary prosthesis of claim 1, wherein said holes are distributed equidistantly or non-equidistantly on said sheet and the distance between two adjacent said holes is from about 5 mm to about 40 mm.

4. The mammary prosthesis of claim 1, wherein said holes are in the shape of a triangle, a circle, a rectangle, a square.

5. The mammary prosthesis of claim 1, wherein said holes have a radius of from about 1 mm to about 10 mm.

6. The mammary prosthesis of claim 5, wherein the radius is from about 2 mm to about 6 mm.

7. A surgical implantable mammary prosthesis adapted to resist capsular contracure, comprising (1) a flexible sac having a generally circular base and containing an appropriate gel or fluid, and (2) a flexible sheet extending outwardly from said circular base and being peripherally integral with said circular base and forming a ring around said circular base, wherein the sheet is made up of 2 or more layers.

8. The mammary prosthesis of claim 7, wherein the width of each layer of said sheet is from about 2 mm to 25 mm.

9. The mammary prosthesis of claim 7, wherein the layers of said sheet are connected with each other.

10. The mammary prosthesis of claim 7, wherein the layers of said sheet are separated from each other.

11. The mammary prosthesis of claim 7, wherein the flexible sheet further comprise a multiplicity of holes.
